(19) United States
(12) Patent Application Publication
(10) Pub. No.: US 2022/0323031 A1
(43) Pub. Date: Oct. 13, 2022

(54) METHOD AND SYSTEM FOR HIGH BIT DEPTH IMAGING

(71) Applicant: SHENZHEN XPECTVISION TECHNOLOGY CO., LTD., Shenzhen (CN)

(72) Inventor: Peiyan CAO, Shenzhen (CN)

(21) Appl. No.: 17/846,361

(22) Filed: Jun. 22, 2022

Related U.S. Application Data
(63) Continuation of application No. PCT/CN2020/071298, filed on Jan. 10, 2020.

(51) Int. Cl. A61B 6/00; G01T 1/29; G06T 7/00

(52) U.S. Cl. CPC A61B 6/502; A61B 6/488; G01T 1/2992; G06T 7/0012; G06T 2207/30068; G06T 2207/10116

(57) ABSTRACT

Disclosed herein is a method comprising: capturing a first image of a tissue using radiation; selecting a region of the tissue based on the first image; capturing a second image of the tissue in the region using the radiation; wherein a signal-to-noise ratio of the second image is higher than a signal-to-noise ratio of the first image.

### BACKGROUND

An image sensor or imaging sensor is a sensor that can detect a spatial intensity distribution of a radiation. An image sensor usually represents the detected image by electrical signals. Image sensors based on semiconductor devices may be classified into several types, including semiconductor charge-coupled devices (CCD), complementary metal-oxide-semiconductor (CMOS), N-type metal-oxide-semiconductor (NMOS). A CMOS image sensor is a type of active pixel sensor made using the CMOS semiconductor process. Light incident on a pixel in the CMOS image sensor is converted into an electric voltage. The electric voltage is digitized into a discrete value that represents the intensity of the light incident on that pixel. An active-pixel sensor (APS) is an image sensor that includes pixels with a photodetector and an active amplifier. A CCD image sensor includes a capacitor in a pixel. When light incidents on the pixel, the light generates electrical charges and the charges are stored on the capacitor. The stored charges are converted to an electric voltage and the electrical voltage is digitized into a discrete value that represents the intensity of the light incident on that pixel.

### SUMMARY

Disclosed herein is a method comprising: capturing a first image of a tissue using radiation; selecting a region of the tissue based on the first image; capturing a second image of the tissue in the region using the radiation; wherein a signal-to-noise ratio of the second image is higher than a signal-to-noise ratio of the first image.

In an aspect, the signal-to-noise ratio of the first image is less than 210.

In an aspect, the signal-to-noise ratio of the second image is greater than 216.

In an aspect, the signal-to-noise ratio of the second image is at least 26 of the signal-to-noise ratio of the first image.

In an aspect, noise in the first image consists of shot noise.

In an aspect, noise in the second image consists of shot noise.

In an aspect, the method further comprises preventing exposure of the tissue outside of the region to the radiation before capturing the second image.

In an aspect, the second image is captured with a higher dose of the radiation than the first image.

In an aspect, the tissue is a human breast tissue.

In an aspect, the radiation is X-ray.

In an aspect, the first image and the second image are captured using an image sensor configured to count numbers of particles of the radiation incident on a plurality of pixels of the image sensor, within a period of time.

In an aspect, the image sensor comprises: a radiation absorption layer comprising an electric contact; a first voltage comparator configured to compare a voltage of the electric contact to a first threshold; a second voltage comparator configured to compare the voltage to a second threshold; a counter configured to register a number of particles of radiation incident on the radiation absorption layer; a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause at least one of the numbers of particles to increase by one, when the second voltage comparator determines that an absolute value of the voltage equals or exceeds an absolute value of the second threshold.

In an aspect, the image sensor does not comprise a scintillator.

Disclosed herein is a computer program product comprising a non-transitory computer readable medium having instructions recorded thereon, the instructions when executed by a computer implementing any of the above method.

Disclosed herein is a system comprising: a radiation source configured to direct radiation to a tissue; a clamp configured to compress the tissue; a mask with a window, the mask configured to adjust a position of the window relative to the clamp and to adjust a size of the window, wherein the radiation is not able to penetrate the mask except within the window; an image sensor; a processor configured: to cause the image sensor to capture a first image of the tissue using the radiation, to select a region of the tissue based on the first image, to cause the mask to adjust the position and the size of the window so that the region is coextensive with the window, and to cause the image sensor to capture a second image of the tissue in the region using the radiation; wherein a signal-to-noise ratio of the second image is higher than a signal-to-noise ratio of the first image.

In an aspect, the signal-to-noise ratio of the first image is less than 210.

In an aspect, the signal-to-noise ratio of the second image is greater than 216.

In an aspect, the signal-to-noise ratio of the second image is at least 26 of the signal-to-noise ratio of the first image.

In an aspect, noise of the first image consists of shot noise.

In an aspect, noise of the second image consists of shot noise.

In an aspect, the second image is captured with a higher dose of the radiation than the first image.

In an aspect, the tissue is a human breast tissue.

In an aspect, the radiation is X-ray.

In an aspect, the image sensor is configured to count numbers of particles of the radiation incident on a plurality of pixels of the image sensor, within a period of time.

In an aspect, the image sensor comprises: a radiation absorption layer comprising an electric contact; a first voltage comparator configured to compare a voltage of the electric contact to a first threshold; a second voltage comparator configured to compare the voltage to a second threshold; a counter configured to register a number of particles of radiation incident on the radiation absorption layer; a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause at least one of the numbers of particles to increase by one, when the second voltage comparator determines that an absolute value of the voltage equals or exceeds an absolute value of the second threshold.

In an aspect, the image sensor does not comprise a scintillator.

### BRIEF DESCRIPTION OF FIGURES

FIG. 1 shows images respectively captured from the same scene by image sensors with bit depths of 8 bits, 4 bits, 2 bits and 1 bit in their pixels.

FIG. 2A schematically shows a flowchart for a method, according to an embodiment.

FIG. 2B shows schematic examples of the first image, the region and the second image involved in the method of FIG. 2A.

FIG. 3 schematically shows a system, according to an embodiment.

FIG. 4 schematically shows that an image sensor may have an array of pixels, according to an embodiment.

FIG. 5A schematically shows a cross-sectional view of the image sensor, according to an embodiment.

FIG. 5B schematically shows a detailed cross-sectional view of the image sensor, according to an embodiment.

FIG. 5C schematically shows an alternative detailed cross-sectional view of the image sensor, according to an embodiment.

FIG. 6A and FIG. 6B schematically show a component diagram of an electronic system of the image sensor, according to an embodiment.

FIG. 7 schematically shows a temporal change of the electric current flowing through an electric contact (upper curve) of the radiation absorption layer of the image sensor, and a corresponding temporal change of the voltage on the electric contact (lower curve).

### DETAILED DESCRIPTION

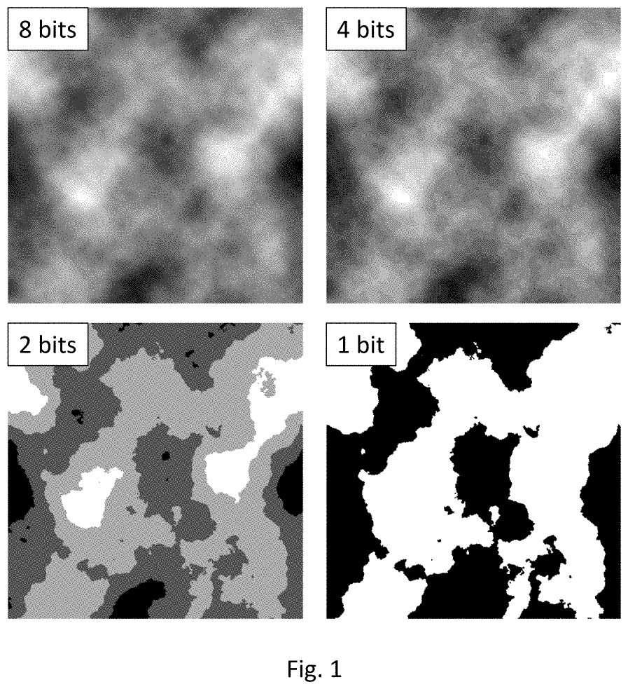

One of the metrics for measuring the performance of an image sensor is the bit depth of its pixels. A pixel with a bit depth B can have 2^{B} distinctive levels within its range R. The range R is the range between the maximum level of the signal the pixel can record and the minimum level of the signal the pixel can record (e.g., zero). For example, a pixel with a bit depth of 8 bits can have 2⁸=256 distinctive levels within its range while a pixel with a bit depth of 1 bit can only have 2¹=2 distinct levels (e.g., black and white). FIG. 1 shows images respectively captured from the same scene by image sensors with bit depths of 8 bits, 4 bits, 2 bits and 1 bit in their pixels. The higher the bit depth is, the finer the differences in the signal resolvable by the pixel are. Assuming the pixel is perfectly linear (i.e., the levels being equally spaced), a pixel with a bit depth B can resolve a difference of at least R/2^{B}. However, the resolution of the pixel (e.g., as represented by R/2^{B}) may be limited by the noise of the pixel. The noise may be in the signal or caused by the pixel. If the noise has a range of R/2^{N}, resolving a difference below R/2^{N} is meaningless because such a difference is masked by the noise. Therefore, the metric more meaningful than the bit depth B is the signal-to-noise ratio (SNR), which can be represented by (B-N).

In certain image sensors, the noise includes the dark noise. The dark noise is the noise present even without the presence of the signal and hence the word “dark” in its name. The dark noise is still present with the presence of the signal. The dark noise may depend on the temperature of these image sensors. Because the dark noise is always present in these image sensors, longer exposure to a scene does not reduce the SNR if the dark noise is the dominant noise. If an image sensor does not suffer from the dark noise (e.g., the image sensor described below, which is able to exclude the dark noise from the signal it records), longer exposure may reduce the SNR.

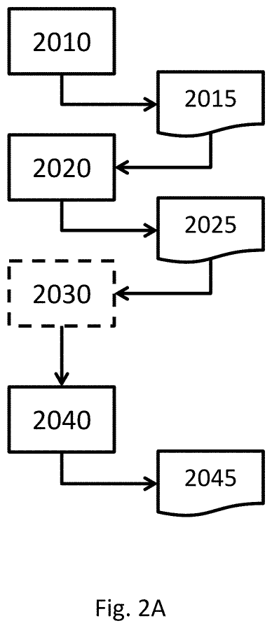

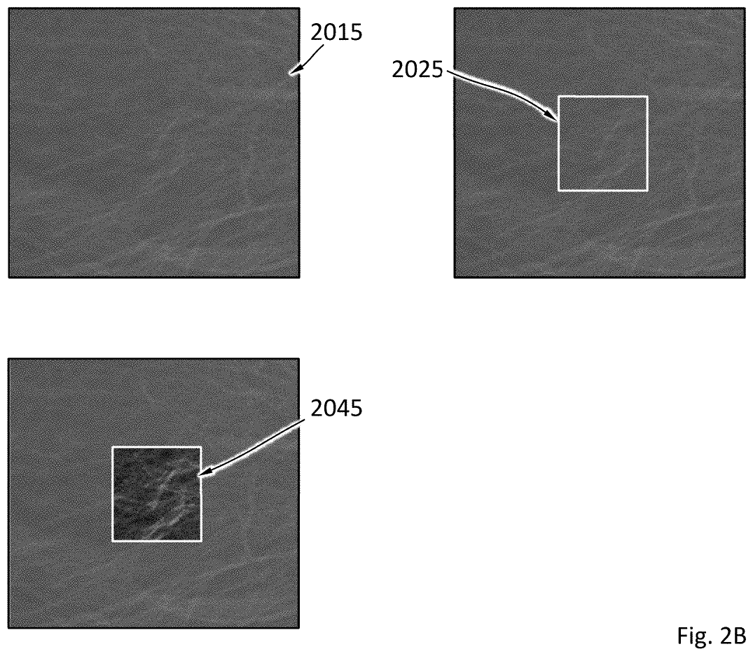

In the application of medical imaging, such as mammography, a subject may be surveyed by capturing an image of the entire subject at a lower SNR, which often uses a lower dose of radiation. If a portion of the subject is difficult to image at the lower SNR, an image of that portion may be captured at a higher SNR. FIG. 2A schematically shows a flowchart for a method. In procedure 2010, a first image 2015 of a tissue is captured using radiation. In an example, the tissue is a human breast tissue. In the example, the radiation is X-ray. The dose of the radiation for capturing the first image 2015 may be limited, for example, by using a short exposure time. When the tissue is a normal soft tissue, a low dose is sufficient. In procedure 2020, a region 2025 of the tissue is selected based on the first image 2015. The region 2025 may be a region that is denser than the rest of the tissue and thus the dose for capturing the first image 2015 is insufficient to provide large enough SNR. The region 2025 may include calcification or overlapping structures (e.g., lobules, glands). In optional procedure 2030, exposure of the tissue outside of the region 2025 to the radiation is prevented before procedure 2040. In an example, a mask opaque to the radiation with a window not opaque to the radiation may be placed upstream from the tissue so that the window only allows that radiation that would incident on the region to pass. The mask may be made of a suitable material such as lead. In procedure 2040, a second image 2045 of the tissue in the region 2025 is captured using the radiation. The SNR of the second image 2045 is higher than the SNR of the first image 2015. The higher SNR of the second image 2045 may be achieved by using a higher dose of the radiation (e.g., increasing the duration of exposure, increasing the intensity of the radiation, or both). In an example, the SNR of the first image 2015 is less than 2¹⁰. In an example, the SNR of the second image 2045 is greater than 2¹⁶. In an example, the SNR of the second image 2045 is at least 2⁶ of the SNR of the first image 2015. FIG. 2B shows schematic examples of the first image 2015, the region 2025 and the second image 2045. In an example, the first image 2015 and the second image 2045 are captured using an image sensor that is able to exclude the dark noise. The noise in the first image 2015 and the second image 2045 may be only shot noise caused by the particle nature of the radiation. Shot noise increases according to the square root of the number of photons incident on a pixel. In other words, shot noise does not increase as fast as the signal increases. Therefore, increasing the dose leads a higher SNR when the images only have shot noise.

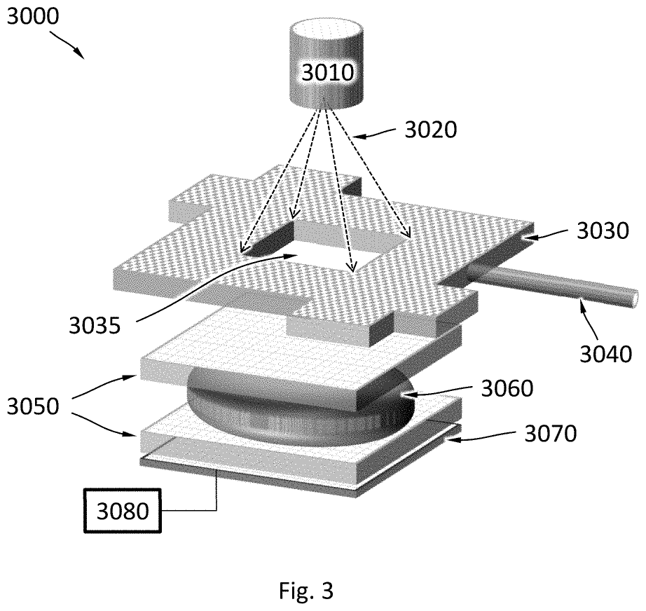

FIG. 3 schematically shows a system 3000. The system 3000 has a radiation source 3010 configured to direct radiation 3020 to a tissue 3060. The system 3000 has a clamp 3050 configured to compress the tissue 3060. The system 3000 also has a mask 3030. The mask 3030 has a window 3035. The mask 3030 is opaque to the radiation 3020 except within the window 3035. Namely, the radiation 3020 cannot go through the mask 3030 except through the window 3035. The size of the window 3035 is adjustable. The position of the window 3035 relative to the clamp 3050 is also adjustable. For example, the system 3000 may have an actuator 3040 configured to move the mask 3030 and adjust the size of the window 3035. The system 3000 has an image sensor 3070 and a processor 3080. The processor 3080 is configured to cause the image sensor 3070 to capture the first image 2015 of the tissue 3060 using the radiation 3020, to select the region 2025 of the tissue 3060 based on the first image 2015, to cause the mask 3030 to adjust the position and the size of the window 3035 so that the region 2025 is coextensive with the window 3035, and to cause the image sensor 3070 to capture a second image 2045 of the tissue 3060 in the region 2025 using the radiation 3020.

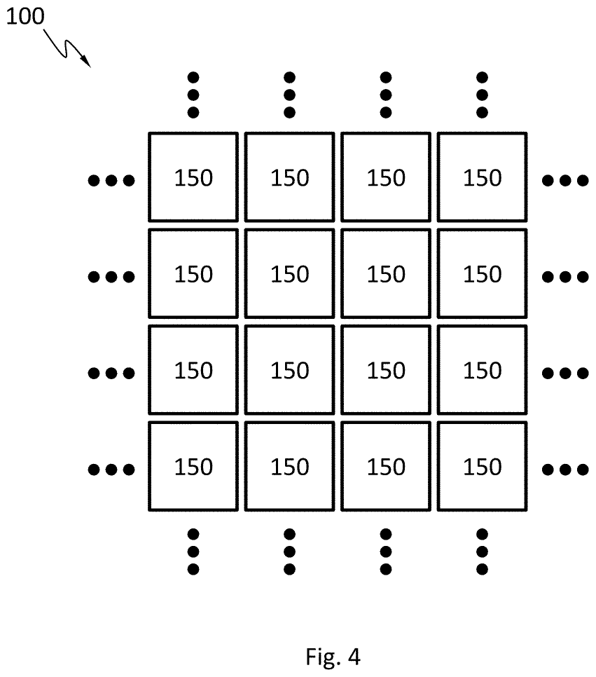

FIG. 4-FIG. 7 schematically show the structure and operation of an image sensor 100 that may be used in the method and system above. FIG. 4 schematically shows that the image sensor 100 may have an array of pixels 150, according to an embodiment. The array of the pixels 150 may be a rectangular array, a honeycomb array, a hexagonal array or any other suitable array. The image sensor 100 may count numbers of particles of radiation incident on the pixels 150, within a period of time. An example of the particles of radiation is X-ray photons. In an example, the X-ray photons have energies between 20 keV and 30 keV. The pixels 150 may be configured to operate in parallel. For example, the image sensor 100 may count one particle of radiation incident on one pixel 150 before, after or while the image sensor 100 counts another particle of radiation incident on another pixel 150. The pixels 150 may be individually addressable.

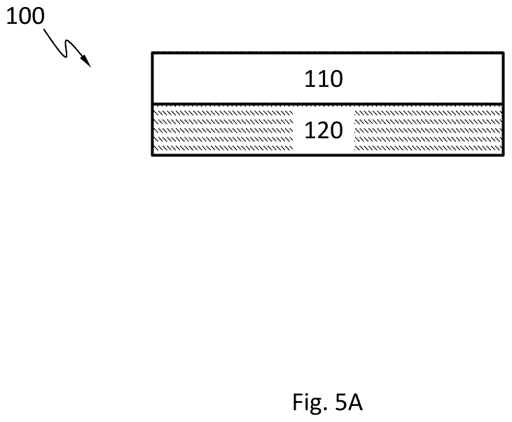

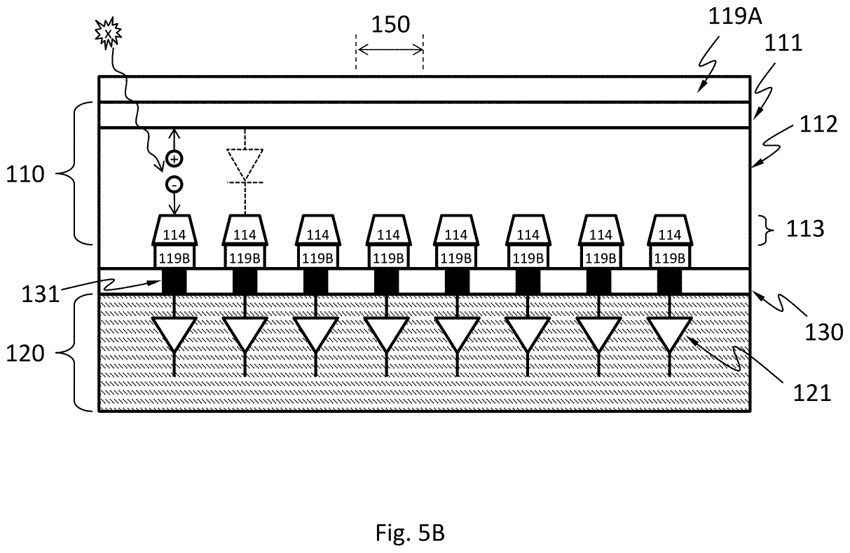

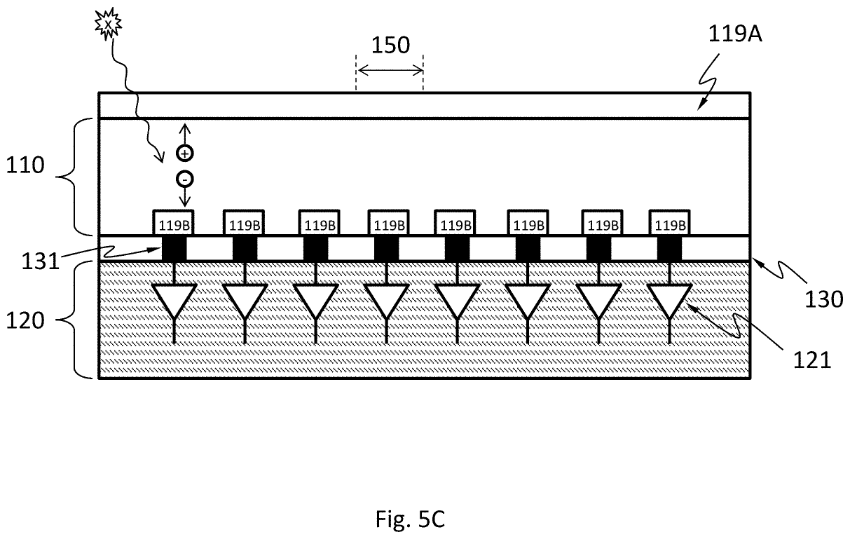

FIG. 5A shows a cross-sectional schematic of the image sensor 100, according to an embodiment. The image sensor 100 may include a radiation absorption layer 110 and an electronics layer 120 (e.g., an ASIC) for processing or analyzing electrical signals incident particles of radiation generate in the radiation absorption layer 110. The image sensor 100 does not include a scintillator. The radiation absorption layer 110 may include a semiconductor material such as single-crystalline silicon. The semiconductor may have a high mass attenuation coefficient for the radiation of interest.

As shown in a more detailed cross-sectional schematic of the image sensor 100 in FIG. 5B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 5B, each of the discrete regions 114 of the second doped region 113 forms a diode with the first doped region 111 and the optional intrinsic region 112. Namely, in the example in FIG. 5B, the radiation absorption layer 110 has a plurality of diodes having the first doped region 111 as a shared electrode. The first doped region 111 may also have discrete portions. The radiation absorption layer 110 may have an electric contact 119A in electrical contact with the first doped region 111. The radiation absorption layer 110 may have multiple discrete electric contacts 119B, each of which is in electrical contact with the discrete regions 114.

When particles of radiation hit the radiation absorption layer 110 including diodes, the particles of radiation may be absorbed and generate one or more charge carriers by a number of mechanisms. The charge carriers may drift to the electric contacts 119A and 119B under an electric field. The field may be an external electric field. In an embodiment, the charge carriers may drift in directions so that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

As shown in an alternative detailed cross-sectional schematic of the image sensor 100 in FIG. 5C, according to an embodiment, the radiation absorption layer 110 may include a resistor of a semiconductor material such as single-crystalline silicon but does not include a diode. The semiconductor may have a high mass attenuation coefficient for the radiation of interest. The radiation absorption layer 110 may have an electric contact 119A in electrical contact with the semiconductor on one surface of the semiconductor. The radiation absorption layer 110 may have multiple electric contacts 119B on another surface of the semiconductor.

When particles of radiation hit the radiation absorption layer 110 including a resistor but not diodes, the particles of radiation may be absorbed and generate one or more charge carriers by a number of mechanisms. A particle of the radiation may generate 10 to 100000 charge carriers. The charge carriers may drift to the electric contacts 119A and 119B under an electric field. The field may be an external electric field. In an embodiment, the charge carriers may drift in directions so that the charge carriers generated by a single particle of the radiation are not substantially shared by two electric contacts 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of the electric contacts 119B are not substantially shared with another of the electric contacts 119B. A pixel 150 associated with one of the electric contacts 119B may be an area around it in which substantially all (more than 98%, more than 99.5%, more than 99.9% or more than 99.99% of) charge carriers generated by a particle of the radiation incident therein flow to that one electric contact 119B. Namely, less than 2 %, less than 0.5%, less than 0.1%, or less than 0.01% of these charge carriers flow beyond the pixel associated with that one electric contact 119B.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels or components dedicated to a single pixel. For example, the electronic system 121 may include an amplifier dedicated to each pixel 150 and a microprocessor shared among all the pixels 150. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

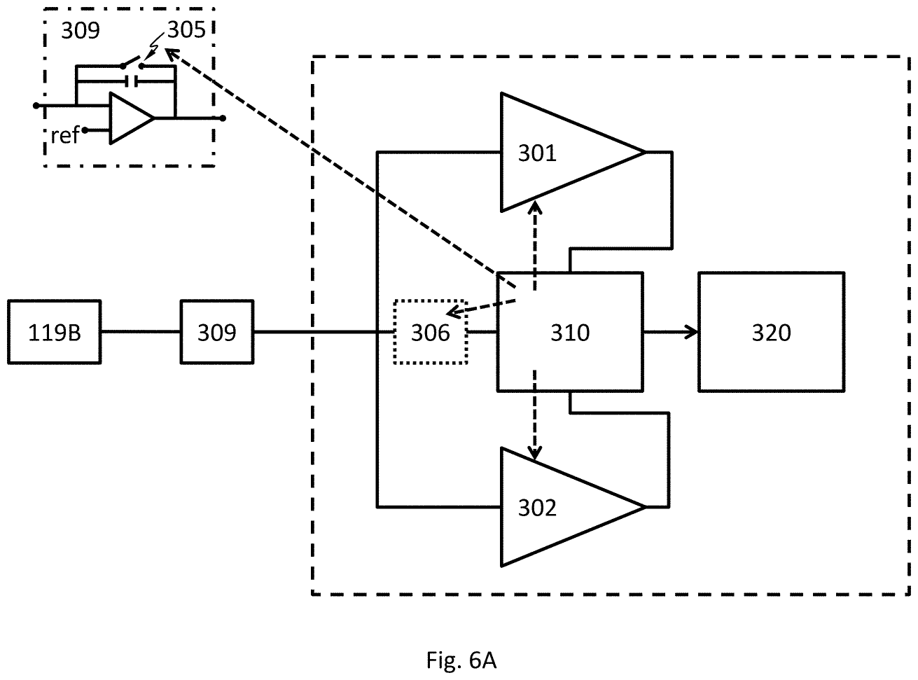

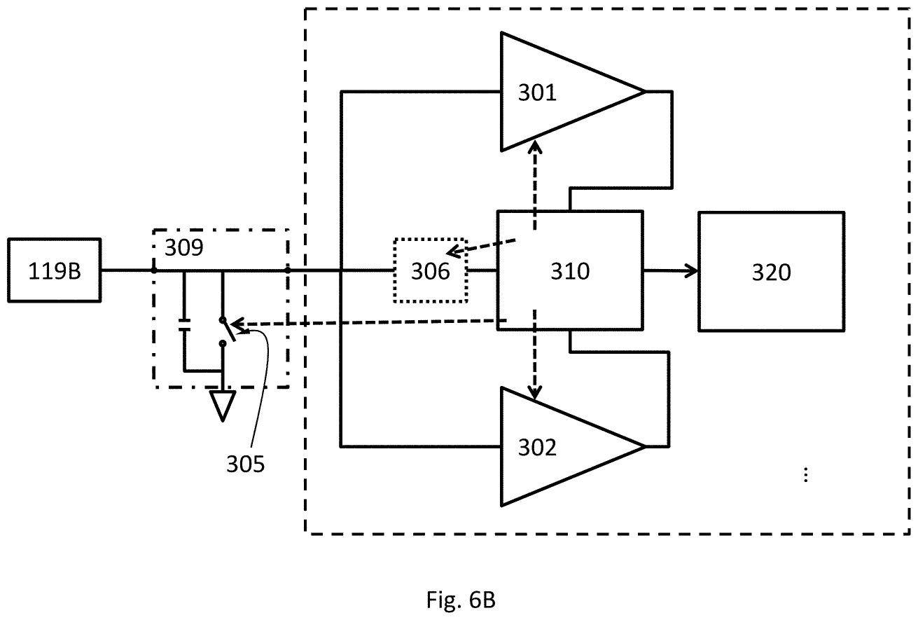

FIG. 6A and FIG. 6B each show a component diagram of the electronic system 121, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or calculate the voltage by integrating an electric current flowing through the electric contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident particle of radiation may generate on the electric contact 119B. The maximum voltage may depend on the energy of the incident particle of radiation, the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electric contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its sign. Namely,

$$\vert x \vert = \{ \begin{matrix}x , if x \geq 0 \\ - x , if x \leq 0\end{matrix} .$$

The second threshold may be 200%-300% of the first threshold. The second threshold may be at least 50% of the maximum voltage one incident particle of radiation may generate on the electric contact 119B. For example, the second threshold may be 100 mV, 150 mV, 200 mV, 250 mV or 300 mV. The second voltage comparator 302 and the first voltage comparator 310 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the system 121 to operate under a high flux of incident particles of radiation. However, having a high speed is often at the cost of power consumption.

The counter 320 is configured to register a number of particles of radiation incident on the radiation absorption layer 110. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electric contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause at least one of the numbers of particles registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In an embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 can include a capacitor directly connected to the electric contact 119B.

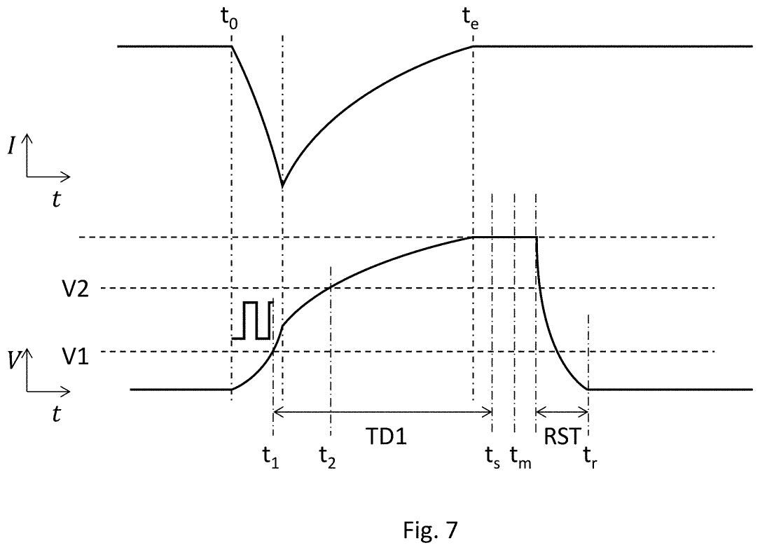

FIG. 7 schematically shows a temporal change of the electric current flowing through the electric contact 119B (upper curve) caused by charge carriers generated by a particle of radiation incident on the pixel 150 encompassing the electric contact 119B, and a corresponding temporal change of the voltage of the electric contact 119B (lower curve). The voltage may be an integral of the electric current with respect to time. At time to, the particle of radiation hits pixel 150, charge carriers start being generated in the pixel 150, electric current starts to flow through the electric contact 119B, and the absolute value of the voltage of the electric contact 119B starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize.

The voltage stabilizes at time tₑ, when all charge carriers generated by the particle of radiation drift out of the radiation absorption layer 110. At time tₛ, the time delay TD1 expires. At or after time tₑ, the controller 310 causes the voltmeter 306 to digitize the voltage and determines which bin the energy of the particle of radiation falls in. The controller 310 then causes the number registered by the counter 320 corresponding to the bin to increase by one. In the example of FIG. 7, time tₛ is after time tₑ; namely TD1 expires after all charge carriers generated by the particle of radiation drift out of the radiation absorption layer 110. If time tₑ cannot be easily measured, TD1 can be empirically chosen to allow sufficient time to collect essentially all charge carriers generated by a particle of radiation but not too long to risk have another incident particle of radiation. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the voltage may be substantially zero at tₑ. The controller 310 may be configured to deactivate the second voltage comparator 302 at expiration of TD1 or at t₂, or any time in between.

The voltage at time tₑ is proportional to the amount of charge carriers generated by the particle of radiation, which relates to the energy of the particle of radiation. The controller 310 may be configured to determine the energy of the particle of radiation, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the system 121 is ready to detect another incident particle of radiation. If the first voltage comparator 301 has been deactivated, the controller 310 can activate it at any time before RST expires. If the controller 310 has been deactivated, it may be activated before RST expires.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method comprising:
capturing a first image of a tissue using radiation;
selecting a region of the tissue based on the first image;
capturing a second image of the tissue in the region using the radiation;
wherein a signal-to-noise ratio of the second image is higher than a signal-to-noise ratio of the first image.

2. The method of claim 1, wherein the signal-to-noise ratio of the first image is less than 2¹⁰.

3. The method of claim 1, wherein the signal-to-noise ratio of the second image is greater than 2¹⁶.

4. The method of claim 1, wherein the signal-to-noise ratio of the second image is at least 2⁶ of the signal-to-noise ratio of the first image.

5. The method of claim 1, wherein noise in the first image consists of shot noise.

6. The method of claim 1, wherein noise in the second image consists of shot noise.

7. The method of claim 1, further comprising preventing exposure of the tissue outside of the region to the radiation before capturing the second image.

8. The method of claim 1, wherein the second image is captured with a higher dose of the radiation than the first image.

9. The method of claim 1, wherein the tissue is a human breast tissue.

10. The method of claim 1, wherein the radiation is X-ray.

11. The method of claim 1, wherein the first image and the second image are captured using an image sensor configured to count numbers of particles of the radiation incident on a plurality of pixels of the image sensor, within a period of time.

12. The method of claim 11, wherein the image sensor comprises:
a radiation absorption layer comprising an electric contact;
a first voltage comparator configured to compare a voltage of the electric contact to a first threshold;
a second voltage comparator configured to compare the voltage to a second threshold;
a counter configured to register a number of particles of radiation incident on the radiation absorption layer;
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause at least one of the numbers of particles to increase by one, when the second voltage comparator determines that an absolute value of the voltage equals or exceeds an absolute value of the second threshold.

13. The method of claim 12, wherein the image sensor does not comprise a scintillator.

14. A computer program product comprising a non-transitory computer readable medium having instructions recorded thereon, the instructions when executed by a computer implementing a method of claim 1.

15. A system comprising:
a radiation source configured to direct radiation to a tissue;
a clamp configured to compress the tissue;
a mask with a window, the mask configured to adjust a position of the window relative to the clamp and to adjust a size of the window, wherein the radiation is not able to penetrate the mask except within the window;
an image sensor;
a processor configured:
to cause the image sensor to capture a first image of the tissue using the radiation,
to select a region of the tissue based on the first image,
to cause the mask to adjust the position and the size of the window so that the region is coextensive with the window, and
to cause the image sensor to capture a second image of the tissue in the region using the radiation;
wherein a signal-to-noise ratio of the second image is higher than a signal-to-noise ratio of the first image.

16. The system of claim 15, wherein the signal-to-noise ratio of the first image is less than 2¹⁰.

17. The system of claim 15, wherein the signal-to-noise ratio of the second image is greater than 2¹⁶.

18. The system of claim 15, wherein the signal-to-noise ratio of the second image is at least 2⁶ of the signal-to-noise ratio of the first image.

19. The system of claim 15, wherein noise of the first image consists of shot noise.

20. The system of claim 15, wherein noise of the second image consists of shot noise.

21. The system of claim 15, wherein the second image is captured with a higher dose of the radiation than the first image.

22. The system of claim 15, wherein the tissue is a human breast tissue.

23. The system of claim 15, wherein the radiation is X-ray.

24. The system of claim 15, wherein the image sensor is configured to count numbers of particles of the radiation incident on a plurality of pixels of the image sensor, within a period of time.

25. The system of claim 24, wherein the image sensor comprises:
a radiation absorption layer comprising an electric contact;
a first voltage comparator configured to compare a voltage of the electric contact to a first threshold;
a second voltage comparator configured to compare the voltage to a second threshold;
a counter configured to register a number of particles of radiation incident on the radiation absorption layer;
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause at least one of the numbers of particles to increase by one, when the second voltage comparator determines that an absolute value of the voltage equals or exceeds an absolute value of the second threshold.

26. The system of claim 25, wherein the image sensor does not comprise a scintillator.
